(19) United States
(12) Patent Application Publication
(10) Pub. No.: US 2017/0340552 A1
(43) Pub. Date: Nov. 30, 2017

(54) Fan Masque

(71) Applicant: Adrienne Austermann, BETHEL, CT (US)

(72) Inventor: Adrienne Austermann, BETHEL, CT (US)

(21) Appl. No.: 15/168,229

(22) Filed: May 30, 2016

(51) Int. Cl. A61K 8/92; A61K 8/27; A61K 8/34; A61Q 1/02; A61K 8/02; A61K 8/97; A61K 8/26; A61Q 17/04

(52) U.S. Cl. CPC A61K 8/922; A61K 8/26; A61K 8/27; A61Q 17/04; A61Q 1/02; A61K 8/0212; A61K 8/97; A61K 8/345; A61K 2800/43; A61K 2800/75; A61K 2800/40

(57) ABSTRACT

Fan Masque is a unique cosmetic paint formula. It is comprised of ingredients found in expensive spa facials that treat the skin, yet is formulated to look like paint, in bright sports team inspired colors. Fan Masque was specifically formulated to solve a the problem that my daughter and other young college women experienced. They could not fully engaging in super fan activities at events such as football games, because traditional face paints often cause skin breakouts and irritation. Fan Masque was designed to function as a face paint and facial all in one, made from only natural and organic ingredients. I experimented for months to find the right ingredients and proportions that make Fan Masque water resistant so it will not run in the rain, yet wash off easily with soap and water. It also contains a natural sunscreen which you do not find in traditional face paints or in spa treatment masks. Fan Mask is the first of a new product category thus needs patent protection for its novelty of use. It goes beyond being a face paint, that won't harm your skin, to one that actually benefits it. Every ingredient was carefully chosen for their already proven benefits to the skin.

### DETAILED DESCRIPTION OF THE INVENTION

Fan Masque is created by adding cosmetic grade mica sometimes combined with an iron oxide, to an opaque cream base that is comprised of organic and natural materials including non-nano zinc oxide, emulsifying way nf, glycerin, kaolin and bentonite clay, caster oil, Thayers uncounted witch hazel with aloe vera formula, shea butter, distilled water.

### Fan Masque Base Recipe—Ingredients and Steps

(in a bowl combine)

¼ cup Distilled Water

1 tbs kaolin clay

1 tbs Bentonite clay

2 tbs Glycerin

2 tbs Non-Nano Zinc Oxide

(High speed mix together)

(In a larger glass bowl combine)

2 tbs—Organic shea butter

2 tbs—Organic vegetable base emulsifying wax

2 tbs—Castor oil

(Melt these ingredients together in microwave or double boiler)

5 tbs—Thayers unscented witch Hazel with aloe vera

(when emulsion mixture cools, add 3 tbs of the witch hazel and high speed mix)

Add clay mixture and the remaining 2 tbs of witch hazel into emulsion

(High sped mix together)

That creates the “paint masque” base which you then add the color to, sample color recipes:

### Bleed Blue Navy

(combine together)

2 tbs base

1½ tsp teas Sapphire mica powder

½ tsp blue iron oxide

1 tsp Glycerin

### Dome Gold

(combine together)

2 tbs base

1 tsp Doubloon gold Mica

1 tsp Sparkling gold Mica

1 tsp Glycerin

### SUMMARY OF THE INVENTION

Fan Masque was created to fill a need and solve a problem. Although inspired by a specific application, the need for young women to feel they can engage in super fan activities without worrying about skin irritations and breakouts, it has become clear that the use of this product will benefit any area traditional face paints are used, sports, theater, festivals, halloween and more.

The difference between Fan Masque and other organic, non-toxic face paints, is that Fan Masque takes a huge leap past decorative, in the fact that it can actually treat and heal irritated skin. Its application is what could be the equivalent of an expensive spa facial. Fan Masque contains ingredients such as bentonite clay that detoxifies and draws impurities out of skin, witch hazel which protects from airborne impurities, as well as a non-nano zinc oxide which protects the skin from harmful sun rays. Face paints do not employ this combination of ingredients. Face paints also tend to smear and smudge easily. Fan Masque dries with minimal transfer to the touch, and because of its special mix of ingredients it is water resistant yet easily washes up with soap and water.

The difference between Fan Masque and other organic, non-toxic spa type clay masques is that typically, clay based masques easily dissolve with water, thus a few drops of rain will dissolve it on your face. Fan Masque is water resistant and will not run in the rain. It is purposely designed to withstand outdoor elements like rain and snow it also contains a sunscreen. Spa masks are designed to apply, dry, and remove in a private setting. They have a more sandy and gritty and uneven texture, when they do dry the finish on your skin is not appealing or stable for use as public body ornamentation. Fan Masque dries with a smooth “paint like” texture that maintains an even color tone.

Fan Masque is novel. It was designed so female or other sensitive skin sports enthusiasts can have a product they feel comfortable with leaving on their skin for hours, while they fully engage in team spirit and fun. It's the better option than applying currently marketed face paint products to their skin which tend to cause breakouts and irritation, smudge easily, run in the rain or are not the right team color. No other product is designed with the dual purpose of providing outdoor body ornamentation and a facial at the same time. Fan Masque will be created in the most popular national, college and local level sport team colors encouraging fan participation for everyone to put their game face on!

1. Fan Masque is a face paint comprising:
the unique ingredients and characteristics of a spa facial;
a sunscreen; and
is water resistant.
